(19) United States
(12) Patent Application Publication
(10) Pub. No.: US 2006/0263399 A1
(43) Pub. Date: Nov. 23, 2006

(54) Preparation for external use on skin

(76) Inventors: Rikako Yasuno, Sakai City (JP); Satoshi Inaoka, Osaka City (JP); Shuichi Tsunetsugu, Kobe City (JP)

Correspondence Address: Patent Department;Choate, Hall & Stewart LLP, Exchange Place, 53 State Street, Boston, MA, 02109, US

(21) Appl. No.: 11/140,080

(22) Filed: May 26, 2005

(30) Foreign Application Priority Data
May 20, 2005 (CA) 2,508,095

(51) Int. Cl. A61K 31/375; A61K 8/49; A61K 31/205

(52) U.S. Cl. 424/401; 514/474; 514/554

(57) ABSTRACT

The present invention has as an object to provide a preparation for external use on skin in which a water-soluble ascorbic acid can be stably dissolved. By blending a low molecular weight betaine in a preparation for external use on skin including a water-soluble ascorbic acid and a glycol ether or a specific polyhydric alcohol, the water-soluble ascorbic acid can be stably dissolved. For this reason, a preparation for external use on skin in which a water-soluble ascorbic acid can be blended in a high concentration can be produced, and therefore, the various effects which ascorbic acid inherently possesses can be effectively exhibited depending on the blending amount thereof. In addition, a preparation which does not give a sticky sensation after applying to the skin, provides a moisturizing sensation, and provides good fitness to the skin, can be produced.

### BACKGROUND OF THE INVENTION

1. Field of the Invention

The present invention relates to a preparation for external use on skin in which a water-soluble ascorbic acid is stably solubilized.

2. Description of Related Art

It is known that ascorbic acid exhibits various effects such as anti-inflammatory effects, effects of ameliorating acne, whitening effects, anti-ageing effects, antioxidation effects, effects of stimulating cells due to acceleration of syntheses for biological components such as collagen and the like, effects of controlling DNA damage or cell disorders of epidermal kerationocytes due to UV, and the like. For this reason, ascorbic acid is widely employed in a preparation for external use on skin in anticipation of the effects described above. However, since ascorbic acid is easily oxidized in the presence of water such as in an aqueous solution, it is necessary to reduce the amount of water in a pharmaceutical preparation. However, there is a problem in that a small amount of water cannot dissolve ascorbic acid sufficiently. For this reason, some methods for stably dissolving ascorbic acid in an aqueous preparation for external use on skin have been studied. For example, it is known that ascorbic acid can be stably dissolved in a solution consisting of not more than 15% of ascorbic acid, 10 to 15% of water, 47 to 55% of ethoxydiglycol, 22 to 29% of propylene glycol, and perfumes (Patent document 1: WO 02/19972).

In addition, a method for stably dissolving ascorbic acid in a preparation for external use on skin which is free from water is also studied. For example, it is reported that ascorbic acid can be stably dissolved in a non-aqueous medium comprising a glycol carrier consisting of propylene glycol and butylene glycol and a stabilizer such as ethoxydiglycol or the like (Patent document 2: WO 00/76547).

On the other hand, a low molecular weight betaine represented by trimethylglycine is widely employed as a humectant in a preparation for external use on skin. For example, it is known that greatly superior effects may be obtained of ameliorating the rough skin by simultaneously employing urea and a low molecular weight betaine (Patent document 3: Japanese Unexamined Patent Application, First Publication No. H09-48720); that percutaneous absorption of a whitening agent is improved by adding a whitening component, a low molecular weight betaine, and a silicone oil (Patent document 4: Japanese Unexamined Patent Application, First Publication No. 2001-89321); and the like. However, there is no information about solubilization of ascorbic acid.

### SUMMARY OF THE INVENTION

The present invention has an object to provide a preparation for external use on skin comprising a water-soluble ascorbic acid in which the water-soluble ascorbic acid can be stably dissolved, and thereby, it is possible to blend the water-soluble ascorbic acid in a high concentration.

As a result of diligent research in order to overcome the problems described above, the present inventors discovered that, by employing a preparation for external use on skin comprising a water-soluble ascorbic acid and a glycol ether or a specific polyhydric alcohol, together with a low molecular weight betaine, the water-soluble ascorbic acid can be stably dissolved and blending the water-soluble ascorbic acid in a high concentration can be performed, and thereby a preparation for external use exhibiting superior effects can be produced.

That is, the present invention relates to preparations for external use on skin described in (1) to (6) shown in the following.
(1) A preparation for external use on skin, comprising (A) ascorbic acid, in an amount of not less than 3% by weight; (B) one or more compounds selected from a glycol, a glycol ether, a glycerol, and a diglycerol; and (C) a low molecular weight betaine.
(2) The preparation for external use on skin described in (1), wherein the low molecular weight betaine is trimethylglycine.
(3) The preparation for external use on skin described in (1) or (2), wherein the total weight of the glycol, the glycol ether, the glycerol, and the diglycerol ranges from 30 to 95% by weight based on the total weight of the preparation for external use on skin.
(4) The preparation for external use on skin described in any one of (1) to (3), further comprising one or more components selected from the group consisting of a whitening component, an anti-inflammatory component, an antibacterial component, a cell stimulating component, an astringent component, an antioxidant component, an anti-ageing component, and a humectant component.
(5) A preparation for external use on skin, comprising (A) a water-soluble ascorbic acid; (B) one or more compounds selected from a glycol, a glycol ether, a glycerol, and a diglycerol; and (C) a solubilizer consisting of a low molecular weight betaine.
(6) A solubilizer for an acidic organocompound consisting of a low molecular weight betaine.

In addition, the present invention involves a method for stabilizing a water-soluble ascorbic acid, described in (7) shown below.
(7) A method for stabilizing a water-soluble ascorbic acid, comprising a step of employing a low molecular weight betaine together with a preparation for external use on skin comprising (A) a water-soluble ascorbic acid and (B) one or more compounds selected from a glycol, a glycol ether, a glycerol, and a diglycerol.

### DETAILED DESCRIPTION OF THE INVENTION

In the specification of the present application, “%” means “% by weight” unless otherwise indicated.

In the present invention, a water-soluble ascorbic acid can be stably solubilized by blending a low molecular weight betaine in a preparation for external use on skin comprising a water-soluble ascorbic acid, a glycol ether or a specific polyhydric alcohol. For this reason, a preparation for external use on skin in which a water-soluble ascorbic acid in a high concentration is stably blended can be produced, and various effects which ascorbic acid essentially possesses can be effectively exhibited by varying the blending amount thereof. In addition, a preparation providing no sticky sensation after application to the skin, providing a moisturizing sensation, and exhibiting comfortable fittability to the skin can be produced.

As the ascorbic acid employed in the present invention, commercially available ascorbic acids as components of a preparation for external use on skin in the field of medicines, quasi drugs, or cosmetics, can be employed, and they are, in general, in the L-form.

In addition, the water-soluble ascorbic acid employed in the present invention is not particularly limited as long as they are employed as components of preparations for external use on skin in the field of medicines, quasi drugs, or cosmetics, in addition to the ascorbic acid described above. Examples thereof include ester derivatives or ether derivatives of ascorbic acid. As specific examples of ester derivatives, mention may be made of phosphoric ester derivatives such as L-ascorbyl monophosphoric esters, L-ascorbyl diphosphoric esters, L-ascorbyl triphosphoric esters, or the like; L-ascorbyl-2-sulfuric ester; and the like. In addition, as examples of ether derivatives, mention may be made of L-ascorbyl-2-glucoside and the like. Among these, L-ascorbic acid, phosphoric esters of L-ascorbic acid, L-ascorbyl-2-sulfuric esters, and L-ascorbyl-2-glucoside, are preferable. In view of high safety with respect to the skin or mucosa and increased effects, L-ascorbic acid, L-ascorbyl monophosphoric esters, and L-ascorbyl-2-glucoside, are, in particular, preferable.

In addition, the water-soluble ascorbic acid may be employed as a pharmaceutically acceptable salt. As examples thereof, mention may be made of, for example, salts with an organic base (for example, salts with a tertiary amine such as a trimethylamine salt, a triethylamine salt, a monoethanolamine salt, a triethanolamine salt, pyridine salt, and the like; basic ammonium salts such as arginine and the like; and the like), salts with an inorganic base (for example, ammonium salts, alkali metal salts such as a sodium salt, a potassium salt, and the like, alkaline earth metal salts such as a calcium salt, a magnesium salt, and the like, aluminum salts, and the like). In particular, preferable salts are a sodium salt, and a potassium salt. As specific examples thereof, mention may be made of sodium ascorbate, sodium ascorbyl monophosphate, sodium ascorbyl diphosphate, sodium ascorbyl triphosphate, sodium ascorbyl-2-sulfate, and the like.

The preparation for external use on skin of the present invention can stably dissolve ascorbic acid by blending a low molecular weight betaine. For this reason, the blending amount of ascorbic acid can preferably range from 3 to 30% by weight with respect to the total weight of the preparation for external use on skin. Within the range described above, the amount can be appropriately selected, depending on the various effects of the water-soluble ascorbic acid which are desired and depending on use of the preparation for external use on skin. In view of effects of the present invention, the amount is more preferably in the range of from 5 to 25% by weight, and is, in particular, preferably in the range of 5 to 20% by weight.

In addition, the preparation for external use on skin of the present invention can stably dissolve a water-soluble ascorbic acid by blending a solubilizer consisting of a low molecular weight betaine. For this reason, the amount of the blended water-soluble ascorbic acid is not particularly limited as long as the effects of the present invention can be exhibited. The amount of the water-soluble ascorbic acid commonly ranges from 0.1 to 30% by weight with respect to the total weight of the preparation for external use on skin. Within the range described above, the amount can be appropriately selected, depending on various effects of the water-soluble ascorbic acid which is desired or depending on use of the preparation for external use on skin. In view of the effects of the present invention, the amount is preferably in the range of from 1 to 25% by weight, and is in particular, preferably in the range of from 5 to 20% by weight.

The glycol employed in the present invention is a diol which is a liquid at 25° C. and is employed as a component of a preparation for external use on skin in the field of medicines, quasi drugs, or cosmetics, and examples of which include, for example, a diol represented by a general formula: CₙH₂ₙ(OH)₂, a condensate of the same diols or two or more kinds of the diols described above, or the like. As specific examples thereof, mention may be made of ethylene glycol, propylene glycol, trimethylene glycol, 1,2-butylene glycol, 1,3-butylene glycol, 2,3-butylene glycol, isoprene glycol, 1,2-pentylene glycol, 1,2-hexylene glycol, octylene glycol, and the like; and as condensates, diethylene glycol, triethylene glycol, tetraethylene glycol, dipropylene glycol,. tripropylene glycol, and the like. Propylene glycol and 1,3-butylene glycol are preferable.

In addition, glycerol and diglycerol employed in the present invention are known compounds which are frequently employed in preparations for external use on skin and the like.

The blending amount of the glycol, glycerol, or diglycerol employed in the present invention is not particularly limited as long as the effects of the present invention can be exhibited, and can be appropriately selected in view of sensations in use on the skin or effects. The amount may commonly range from 1 to 95% by weight, may preferably range from 5 to 90% by weight, may more preferably range from 10 to 80% by weight, and in particular, may preferably range from 15 to 65% by weight.

Glycol ether employed in the present invention is a compound in which one or two of the hydroxyl groups of glycol are etherified, and is hot particularly limited as long as it is employed as a component of a preparation for external use on skin in the field of medicines, quasi drugs, or cosmetics. The glycol is a diol which is liquid at 25° C. Examples of glycols include, for example, a diol represented by a general formula: CₙH₂ₙ(OH)₂, a condensate of the same diols or two or more kinds of the diols described above, or the like. As examples thereof, mention may be made of ethylene glycol monoether, ethylene glycol diether, diethylene glycol monoether, diethylene glycol diether, triethylene glycol monoether, triethylene glycol diether, tetraethylene glycol monoether, tetraethylene glycol diether, propylene glycol monoether, propylene glycol diether, dipropylene glycol monoether, dipropylene glycol diether, tripropylene glycol monoether, tripropylene glycol diether, butylene glycol monoether, butylene glycol diether, alkylene glycol ether acetate, and the like. In addition, as examples of ethers, mention may be made of alkyl ethers, alkoxy ethers, aromatic ethers, allyl ethers, and the like. As specific examples thereof, mention may be made of methyl ether, ethyl ether, n-propyl ether, isopropyl ether, n-butyl ether, isobutyl ether, t-butyl ether, n-pentyl ether, n-hexyl ether, phenyl ether, vinyl ether, and the like.

As specific examples of glycol ethers, mention may be made of ethylene glycol monoethers such as ethylene glycol monovinyl ether, ethylene glycol monoethyl ether, ethylene glycol mono-n-propyl ether, ethylene glycol monoisopropyl ether, ethylene glycol mono-n-butyl ether, ethylene glycol mono-isobutyl ether, ethylene glycol mono-t-butyl ether, ethylene glycol mono-2-methylpentyl ether, ethylene glycol mono-n-hexyl ether, ethylene glycol mono-2,4-hexadiene ether, ethylene glycol mono-2,6,8-trimethyl-4-nonyl ether, ethylene glycol monophenyl ether, and ethylene glycol monomethylphenyl ether, and the like; ethylene glycol diethers such as ethylene glycol dimethyl ether, ethylene glycol diethyl ether, and the like; diethylene glycol monoethers such as diethylene glycol monomethyl ether, diethylene glycol monoethyl ether, diethylene glycol mono-n-propyl ether, diethylene glycol mono-n-butyl ether, diethylene glycol monoisobutyl ether, diethylene glycol mono-n-hexyl ether, diethylene glycol ethyl vinyl ether, diethylene glycol monomethyl phenyl ether, and the like; diethylene glycol diethers such as diethylene glycol dimethyl ether, diethylene glycol divinyl ether, and the like; triethylene glycol monoethers such as triethylene glycol monomethyl ether, triethylene glycol monoethyl ether, triethylene glycol mono-n-butyl ether, triethylene glycol monovinyl ethyl ether, and the like; triethylene glycol diethers such as triethylene glycol dimethyl ether, triethylene glycol divinyl ether, and the like; tetraethylene glycol monoethers such as tetraethylene glycol monophenyl ether, and the like; tetraethylene glycol diethers such as tetraethylene glycol diethyl ether, and the like; propylene glycol monoethers such as propylene glycol monomethyl ether, propylene glycol monoethyl ether, propylene glycol mono-n-propyl ether, propylene glycol monoisopropyl ether, propylene glycol mono-n-butyl ether, propylene glycol monoisobutyl ether, propylene glycol monoallyl ether, propylene glycol monophenyl ether, and the like; propylene glycol diethers such as propylene glycol dimethyl ether, propylene glycol diethyl ether, propylene glycol di-n-propyl ether, propylene glycol diisopropyl ether, propylene glycol di-n-butyl ether, propylene glycol diisobutyl ether, propylene glycol diallyl ether, propylene glycol diphenyl ether, and the like; dipropylene glycol monoethers such as dipropylene glycol monoethyl ether, dipropylene glycol mono-n-butyl ether, dipropylene glycol monoisobutyl ether, dipropylene glycol ally ether, and the like; dipropylene glycol diethers such as dipropylene glycol diethyl ether, dipropylene glycol di-n-butyl ether, dipropylene glycol diisobutyl ether, dipropylene glycol allyl ether, and the like; tripropylene glycol monoethers such as tripropylene glycol monomethyl ether, tripropylene glycol monoethyl ether, tripropylene glycol mono-n-butyl ether, tripropylene glycol monoisobutyl ether, tripropylene glycol monoallyl ether, and the like; tripropylene glycol diethers such as tripropylene glycol dimethyl ether, tripropylene glycol diethyl ether, tripropylene glycol di-n-butyl ether, tripropylene glycol diisobutyl ether, tripropylene glycol diallyl ether, and the like; butylene glycol monoethers such as butylene glycol monomethyl ether, butylene glycol monoethyl ether, butylene glycol mono-n-butyl ether, and the like; butylene glycol diethers such as butylene glycol dimethyl ether, butylene glycol diethyl ether, butylene glycol di-n-butyl ether, and the like; alkylene glycol ether acetates such as ethylene glycol monomethyl ether acetate, ethylene glycol monoethyl ether acetate, ethylene glycol mono-n-butyl ether acetate, diethylene glycol monoethyl ether acetate, diethylene glycol mono-n-butyl ether acetate, propylene glycol monomethyl ether acetate, and the like; and the like.

Preferable glycol ethers are ethylene glycol monoether, ethylene glycol diether, diethylene glycol monoether, diethylene glycol diether, triethylene glycol monoether, tetraethylene glycol monoether, tetraethylene glycol diether, propylene glycol monoether, propylene glycol diether, dipropylene glycol monoether, dipropylene glycol diether, and tripropylene glycol monoether. As specific examples thereof, mention may be made of ethylene glycol monovinyl ether, ethylene glycol monoethyl ether, ethylene glycol mono-n-propyl ether, ethylene glycol monoisopropyl ether, ethylene glycol mono-n-butyl ether, ethylene glycol monoisobutyl ether, ethylene glycol mono-t-butyl ether, ethylene glycol mono-2-methylpentyl ether, ethylene glycol dimethyl ether, ethylene glycol diethyl ether, diethylene glycol monomethyl ether, diethylene glycol monoethyl ether, diethylene glycol mono-n-propyl ether, diethylene glycol mono-n-butyl ether, diethylene glycol monoisobutyl ether, diethylene glycol mono-n-hexyl ether, diethylene glycol monomethyl phenyl ether, diethylene glycol dimethyl ether, diethylene glycol divinyl ether, diethylene glycol ethyl vinyl ether, triethylene glycol monomethyl ether, triethylene glycol monoethyl ether, triethylene glycol mono-n-butyl ether, triethylene glycol monovinyl ethyl ether, tetraethylene glycol monophenyl ether, tetraethylene glycol diethyl ether, propylene glycol monomethyl ether, propylene glycol monoethyl ether, propylene glycol mono-n-propyl ether, propylene glycol monoisopropyl ether, propylene glycol mono-n-butyl ether, propylene glycol phenyl ether, propylene glycol monomethyl ether, propylene glycol dimethyl ether, propylene glycol diethyl ether, propylene glycol di-n-propyl ether, propylene glycol diisopropyl ether, propylene glycol di-n-butyl ether, propylene glycol diisobutyl ether, propylene glycol diallyl ether, propylene glycol diphenyl ether, dipropylene glycol monoethyl ether, dipropylene glycol mono-n-butyl ether, dipropylene glycol diethyl ether, and dipropylene glycol di-n-butyl ether, dipropylene glycol diisobutyl ether, dipropylene glycol allyl ether, tripropylene glycol monomethyl ether, tripropylene glycol monoethyl ether, and tripropylene glycol monobutyl ether.

As glycol ethers, ethylene glycol monoether, propylene glycol monoether, and dipropylene glycol monoether are more preferable. As specific examples thereof, mention may be made of ethylene glycol monomethyl ether, ethylene glycol monoethyl ether, ethylene glycol monopropyl ether, diethylene glycol monomethyl ether, diethylene glycol monoethyl ether, diethylene glycol monopropyl ether, diethylene glycol monobutyl ether, propylene glycol monoethyl ether, propylene glycol monopropyl ether, dipropylene glycol monoethyl ether, and dipropylene glycol monopropyl ether.

The blending amount of the glycol ether employed in the present invention is not particularly limited as long as the effects of the present invention can be exhibited, and can be appropriately selected in view of effects and sensations in use on the skin. The amount may commonly range from 1 to 95% by weight, may preferably range from 5 to 90% by weight, may more preferably range from 10 to 80% by weight, and in particular, may preferably range from 15 to 65% by weight with respect to the total weight of the preparation for external use on skin.

The glycols, glycol ethers, glycerols, and diglycerols described above can be employed alone or in combination of two or more kinds thereof. The total amount of a glycol, a glycol ether, a glycerol, and a diglycerol, may preferably range from 30 to 95% by weight, may more preferably range from 40 to 90% by weight, may further more preferably range from 45 to 80% by weight, and may, in particular, preferably range from 50 to 75% by weight, with respect to the total weight of the preparation for external use on skin. The amount is not particularly limited as long as the effects of the present invention can be exhibited.

In the present invention, a low molecular weight betaine means one having a molecular weight of 200 or less, and forming an amphoteric ion. As examples thereof, mention may be made of a quaternary ammonium base, a quaternary phosphonium base, a tertiary sulfonium base, and the like. They exhibit little surfactant activity. Among these, an N,N,N-trialkylamino acid represented by formula (1) shown below is preferable.

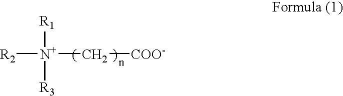

wherein R₁, R₂, and R₃ independently represent an alkyl group having 1 to 6 carbon atoms; and n represents 1 to 6.

As R₁ to R₃, a straight or branched chain alkyl group having 1 to 6 carbon atoms can be widely employed. That is, as examples thereof, mention may be made of, a methyl group, an ethyl group, a propyl group, an isopropyl group, a butyl group, an isobutyl group a sec-butyl group, tert-butyl group, a pentyl group, an isopentyl group, a neopentyl group, a tert-pentyl group, a hexyl group, an isohexyl group, a 3-methylpentyl group, 2,2-dimethylbutyl group, a 2,3-dimethylbutyl group, and the like. R₁ to R₃ may be the same or different.

In particular, in the case of n=1, examples thereof include trimethylglycine, triethylglycine, tripropylglycine, and triisopropylglycine; in the case of n=2, examples thereof include trimethyl-beta-alanine; and in the case of n=3, examples thereof include trimethyl-gamma-aminobutyric acid, and the like. Trimethylglycine is preferable.

In addition, the low molecular weight betaines described above may have substituents. In particular, in the case of n=1, as examples thereof, mention may be made of N,N,N-trimethylalanine, N,N,N-triethylalanine, N,N,N-triisopropylalanine, N,N,N-trimethylmethylalanine, carnitine, acetyl carnitine, and the like. Carnitine is preferable.

In the preparation for external use on skin of the present invention, the blending amount of the low molecular weight betaine preferably ranges from 0.5 to 10% by weight with respect to the total weight of the preparation for external use on skin in order to stably dissolve not less than 3% of ascorbic acid by blending the low molecular weight betaine. Within the range described above, the amount can be appropriately selected depending on desirable various effects of ascorbic acid or depending on use of the preparations for external use on skin. In view of the effects of the present invention, the amount is preferably in the range of from 0.5 to 9% by weight, and is, in particular, preferably in the range of from 1 to 8% by weight. If the amount is below 0.1% by weight, the effects cannot be exhibited in some cases. On the other hand, if the amount exceeds 10% by weight, a poor sensation in use may be provided in some cases.

In addition, in the preparation for external use on skin of the present invention, the blending amount of the solubilizer consisting of a low molecular weight betaine is not particularly limited as long as the water-soluble ascorbic acid- can be stably dissolved. The amount commonly ranges from 0.01 to 10% by weight with respect to the total weight of the preparation for external use on skin. Within the range described above, the amount can be appropriately selected depending on various desirable effects of the water-soluble ascorbic acid and depending on use of the preparations for external use on skin. In view of the effects of the present invention, the amount is preferably in the range of from 0.1 to 9% by weight, and is, in particular, in the range of from 1 to 8% by weight. The solubilizer consisting of the low molecular weight betaine is blended in order to dissolve the water-soluble ascorbic acid.

Furthermore, the low molecular weight betaine can be employed as a solubilizer not only for a water-soluble ascorbic acid, but also for various acidic organic compounds. In this case, one or more kinds of any of a glycol, a glycol ether, a glycerol, and a diglycerol are preferably included.

The preparation for external use on skin of the present invention can exhibit the effects of the invention without blending water. However, by blending water therein, controlling of deposition of the water-soluble ascorbic acid can be enhanced.

The blending ratio of water in the preparation for external use on skin of the present invention may be not more than 2 parts by weight with respect to one part by weight of the water-soluble ascorbic acid in view of instability of the water-soluble ascorbic acid in an aqueous solution. It is preferably not more than 1.5 parts by weight, and is more preferably not more than 1 part by weight. If the ratio exceeds 2 parts by weight, decomposition of the water-soluble ascorbic acid tends to be unacceptable.

The blending amount of water in the preparation for external use on skin of the present invention is not particularly limited as long as the effects of the present invention can be exhibited. It ranges from 5 to 35% by weight with respect to the total weight of the preparation for external use on skin. Within the range, the amount may be appropriately selected. In view of stability of the water-soluble ascorbic acid, it preferably ranges from 5 to 20% by weight, and in particular, preferably ranges from 8 to 13% by weight.

In the preparation for external use on skin of the present invention, in addition to the water-soluble ascorbic acid described above, various components such as a whitening component, an anti-inflammatory component, an antibacterial component, a cell stimulating component, an astringent component, an antioxidant component, a component for ameliorating acne, an anti-ageing component, a component for accelerating syntheses for biological ingredients such as collagen, a blood circulation accelerator component, a humectant component, an anti-ageing component [sic], and the like, can be blended alone or in combination of two or more kinds thereof in order to enhance or assist the various effects which the water-soluble ascorbic acid possesses or in order to add other useful effects to the preparation for external use on skin. Preferably components are one or more kinds of the whitening component, the anti-inflammatory component, the antibacterial component, the cell stimulating component, the astringent component, the antioxidant component, the anti-ageing component, and the humectant component. As examples of the particularly preferable combinations of these components described above, mention may be made of a combination between the water-soluble ascorbic acid and the whitening component, a combination of the water-soluble ascorbic acid and the whitening component and the antioxidant component, a combination between the water-soluble ascorbic acid and the antioxidant component, a combination between the water-soluble ascorbic acid and the anti-ageing component, and a combination of the water-soluble ascorbic acid and the whitening component and the anti-ageing component. The components described above are not particularly limited as long as they are conventionally employed or will be employed in the future as the components of preparations for external use on skin in the field of medicines, quasi drugs, or cosmetics. As the components described above, any components can be appropriately selected and be employed.

For example, as examples of whitening components, mention may be made of arbutin; ellagic acid; phytic acid; rucinol; chamomile ET; vitamins such as vitamin A or derivatives thereof, vitamin E or derivatives thereof, pantothenic acid or derivatives thereof, and the like; and the like. Among these, as preferable examples thereof, mention may be made of pantothenic acid or derivatives thereof, ellagic acid, phytic acid, vitamin A or derivatives thereof, and vitamin E or derivatives thereof. The whitening components described above can be employed alone or in combination of two or more kinds thereof.

Plant components exhibiting whitening effects may be employed as whitening components. As examples of the plant components described above, mention may be made of components derived from plants such as iris, almond, aloe, gingko, oolong tea, rose hips, Scutellaria baicalensis, Coptis japonica, Hypericum erectum, dead nettle, seaweed, Pueraria lobata, cape jasmine, Sophora flavescens, chlorella, Schlechtendaria chinensis, wheat, rice, rice germ, oryzanol, rice bran, Asarum sieboldii, Zanthoxyli fructus, perilla, Paeoniae radix, Cnidium officinale, Morus australis, soybean, fermented soybean, tea, Angelica sinensis, Calendula officinalis, garlic, Hamamelis virginiana, safflower, Paeonia suffruticosa, Coix lacryma-jobi, Angelica sinensis [sic], Salvia leucantha, Uncaria gambir, asebiwarabi [phonetic spelling], Podocarpus macrophyllus, Flammulina velutipes, Diospyros kaki, Catalpa ovata, black bean, Gentiana amarella, Scrophularia buergeriana, Smilax medoca, snap bean, shokuma [phonetic spelling], Paris polyphylla, sage, Peuceadanum praeruptorum, Japanese radish, Ericaceae, Lespedeza homoloba, toshin [phonetic spelling], Picrasma quassioides, parsley, holly, hop, Lespedeza cyrtobotrya, clove, Glycyrrhiza glabra, and the like. Preferable are plant components derived from iris, aloe, gingko, oolong tea, rose hips, Scutellaria baicalensis, Coptis japonica, Hypericum erectum, dead nettle, seaweed, Pueraria lobata, cape jasmine, Sophora flavescens, Schlechtendaria chinensis, wheat, rice, rice bran, Asarum sieboldii, Zanthoxyli fructus, perilla, Paeoniae radix, Cnidium officinale, Morus australis, tea, Angelica sinensis, Calendula officinalis, Hamamelis virginiana, safflower, Paeonia suffruticosa, Coix lacryma-jobi, Salvia leucantha, Uncaria gambir, Flammulina velutipes, Diospyros kaki, Catalpa ovata, black bean, Gentiana amarella, Smilax medoca, snap bean, Paris polyphylla, sage, Peuceadanum praeruptorum, Japanese radish, Ericaceae, Lespedeza homoloba, toshin [phonetic spelling], Picrasma quassioides, parsley, holly, hop, clove, Glycyrrhiza glabra, and Angelica sinensis [sic]. More preferable are plant components derived from iris, aloe, gingko, rose hips, Scutellaria baicalensis, Coptis japonica, Hypericum erectum, cape jasmine, Sophora flavescens, rice, rice bran, Asarum sieboldii, Paeoniae radix, Cnidium officinale, Morus australis, tea, Angelica sinensis, Calendula officinalis, Hamamelis virginiana, safflower, Paeonia suffruticosa, Salvia leucantha, Uncaria gambir, Flammulina velutipes, Diospyros kaki, sage, Japanese radish, Ericaceae, parsley, hop, Glycyrrhiza glabra, and Coix lacryma-jobi. In the case of employing the plant components described above in the preparation for external use on skin of the present invention, the form of the plant components is not particularly limited. In general, the form such as a plant extract, an essential oil, or the like, can be employed.

In the case of employing the whitening components described above, the blending ratio thereof in the preparation for external use on skin of the present invention preferably ranges from 0.0003 to 10% by weight, and more preferably ranges from 0.01 to 5% by weight. In addition, it is preferable that the whitening component be blended in the range of from 0.001 to 1000 parts by weight, preferably in the range of from 0.005 to 500 parts by weight, and more preferably in the range of from 0.01 to 100 parts by weight with respect to 100 parts by weight of the water-soluble ascorbic acid contained in the preparation for external use on skin of the present invention.

In the case of employing the plant components exhibiting whitening effects as the whitening component, one or more kinds thereof can be freely combined and be employed, depending on the purposes. In the case of employing the plant components described above as the whitening component, the blending ratio thereof in the preparation for external use on skin of the present invention, based on the extracted product such as an extract or an essential oil, commonly ranges from 0.00001 to 20% by weight, preferably ranges from 0.0001 to 15% by weight, and more preferably ranges from 0.001 to 10%. by weight. In addition, it is preferable that the plant component be blended in the ratio of from 0.0001 to 100 parts by weight, and preferably in the ratio of from 0.001 to 50 parts by weight with respect to 100 parts by weight of the water-soluble ascorbic acid.

As examples of anti-inflammatory components, mention may be made of allantoin, calamine, glycyrrhizic acid or derivatives thereof, glycyrrhetic acid or derivatives thereof, zinc oxide, guaiazulene, tocopherol acetate, pyridoxine hydrochloride, menthol, camphor, turpentine oil, indomethacin, salicylic acid or derivatives thereof, and the like. Preferable are allantoin, glycyrrhizic acid or derivatives thereof, glycyrrhetic acid or derivatives thereof, guaiazulene, and menthol.

In the case of employing the anti-inflammatory components described above, the ratio thereof blended in the preparation for external use on skin of the present invention preferably ranges from 0.0003 to 10% by weight, and more preferably ranges from 0.01 to 5% by weight. In addition, it is preferable that the anti-inflammatory components be blended in an amount preferably ranging from 0.001 to 1000 parts by weight with respect to 100 parts by weight of the water-soluble ascorbic acid contained in the preparation for external use on skin of the present invention, and more preferably ranging from 0.01 to 100 parts by weight.

As examples of antibacterial components, mention may be made of chlorhexidine, salicylic acid, benzalkonium chloride, acrinol, ethanol, benzethonium chloride, cresol, gluconic acid and derivatives thereof, povidone iodine, potassium iodide, iodine, isopropyl methylphenol, triclocarban, triclosan, sensitizing dye No. 101, sensitizing dye 201, paraben, phenoxyethanol, 1,2-pentane diol, alkyldiaminoglycine hydrochloride, and the like. As preferable examples thereof, mention may be made of benzalkonium chloride, benzethonium chloride, gluconic acid and derivatives thereof, isopropyl methylphenol, triclocarban, triclosan, sensitizing dye No. 101, sensitizing dye No. 201, paraben, phenoxyethanol, 1,2-pentane diol, alkyldiaminoglycine hydrochloride, and the like. More preferable are benzalkonium chloride, gluconic acid and derivatives thereof, benzethonium chloride, and isopropyl methylphenol.

In the case of the antibacterial components described above, the ratio thereof blended in the preparation for external use on skin of the present invention preferably ranges from 0.0003 to 10% by weight, and more preferably ranges from 0.01 to 5% by weight. In addition, it is preferable that the antibacterial components be blended in an amount preferably ranging from 0.001 to 1000 parts by weight with respect to 100 parts by weight of the water-soluble ascorbic acid contained in the preparation for external use on skin of the present invention, and more preferably ranging from 0.005 to 500 parts by weight, and in particular, preferably ranging from 0.01 to 100 parts by weight.

As examples of cell stimulating components, mention may be made of amino acids such as y-aminobutyric acid, ε-aminopuronic acid, and the like; vitamins such as retinol, thiamine, riboflavin, pyridoxine hydrochloride, pantothenic acid, and the like; alpha-hydroxylic acids such as glycolic acid, lactic acid, and the like; tannin, flavonoid, saponin, allatoin, sensitizing dye No. 301, and the like. Preferable are amino acids such as γ-aminobutyric acid, ε-aminopuronic acid, and the like; and vitamins such as retinol, thiamine, riboflavin, pyridoxine hydrochloride, pantothenic acid, and the like.

In the case of employing the cell stimulating components described above, the ratio thereof blended in the preparation for external use on skin of the present invention preferably ranges from 0.0003 to 10% by weight, and more preferably ranges from 0.01 to 5% by weight. In addition, it is preferable that the cell stimulating components be blended in an amount preferably ranging from 0.001 to 1000 parts by weight with respect to 100 parts by weight of the water-soluble ascorbic acid contained in the preparation for external use on skin of the present invention, and more preferably ranging from 0.005 to 500 parts by weight, and in particular, preferably ranging from 0.01 to 1.00 parts by weight.

As examples of astringent components, mention may be made of metal salts such as alum, chlorohydroxyaluminum, aluminum chloride, allantoin aluminum salt, zinc sulfate, aluminum potassium sulfate, and the like; and organic acids such as tannic acid, citric acid, lactic acid, succinic acid, and the like. Preferable are alum, chlorohydroxyaluminum, aluminum chloride, allantoin aluminum salt, aluminum potassium sulfate, and tannic acid.

In the case of employing the astringent components described above, the ratio thereof blended in the preparation for external use on skin of the present invention commonly ranges from 0.0003 to 10% by weight, preferably ranges from 0.01 to 5% by weight, and more preferably ranges from 0.01 to 5% by weight [sic]. In addition, it is preferable that the astringent components be blended in an amount preferably ranging from 0.001 to 1000 parts by weight with respect to 100 parts by weight of the water-soluble ascorbic acid contained in the preparation for external use on skin of the present invention, and more preferably ranging from 0.005 to 500 parts by weight, and in particular, preferably ranging from 0.01 to 100 parts by weight.

As examples of antioxidant components, mention may be made of tocopherol and derivatives thereof, butylhydroxyanisole, dibutylhydroxytoluene, sodium hydrogen sulfite, erythorbic acid and salts thereof, flavonoid, glutathione, glutathione peroxidase, glutathione-S-transferase, catalase, superoxide dismutase, thioredoxin, taurine, thiotaurine, hypotaurine, and the like. Preferable are tocopherol and derivatives thereof, thiotaurine, hypotaurine, thioredoxin, and flavonoid.

In the case of employing the antioxidant components described above, the ratio thereof blended in the preparation for external use on skin of the present invention commonly ranges from 0.00001 to 10% by weight, preferably ranges from 0.0001 to 5% by weight, and more preferably ranges from 0.001 to 5% by weight. In addition, it is preferable that the antioxidant components be blended in an amount preferably ranging from 0.001 to 1000 parts by weight with respect to 100 parts by weight of the water-soluble ascorbic acid contained in the preparation for external use on skin of the present invention, and more preferably ranging from 0.005 to 500 parts by weight, and in particular, preferably ranging from 0.01 to 100 parts by weight.

As examples of anti-ageing components, mention may be made of retinoid (retinol, retinoic acid, retinal, and the like), pangamic acid, kinetin, ursolic acid, an extract of Curcuma longa, sphingosine derivatives, silicon, silicic acid, N-methyl-L-serine, mevalonolactone, and the like. Preferable are retinoid (retinol, retinoic acid, retinal, and the like), and kinetin.

In the case of employing the anti-ageing components described above, the ratio thereof blended in the preparation for external use on skin of the present invention preferably ranges from 0.0003 to 10% by weight, and more preferably ranges from 0.01 to 5% by weight. In addition, it is preferable that the anti-ageing components be blended in an amount preferably ranging from 0.001 to 1000 parts by weight with respect to 100 parts by weight of the water-soluble ascorbic acid contained in the preparation for external use on skin of the present invention, and more preferably ranging from 0.005 to 500 parts by weight, and in particular, preferably ranging from 0.01 to 100 parts by weight.

As examples of humectant components, mention may be made of amino acids and derivatives thereof such as alanine, serine, leucine, isoleucine, threonine, glycine, proline, hydroxyproline, glucosamine, theanine, and the like; peptides such as collagen, collagen peptide, gelatin, and the like; polyhydric alcohols such as glycerol, 1,3-butylene glycol, propylene glycol, polyethylene glycol, and the like; sugar alcohols such as sorbitol and the like; phospholipids such as lecithin, hydrogenated lecithin, and the like; mucopolysaccharides such as hyaluronic acid, heparin, chondroitin, and the like; components based on NMF such as lactic acid, sodium pyrrolidone carbonate, urea, and the like; polyglutamic acid, and the like. Preferable are alanine, serine, glycine, proline, hydroxyproline, glucosamine, theanine, collagen, collagen peptide, glycerol, 1,3-butylene glycol, hydrogenated lecithin, hyaluronic acid, heparin, chondroitin, lactic acid, sodium pyrrolidone carbonate, and polyglutamic acid.

In the case of employing humectant components, the ratio thereof blended in the preparation for external use on skin of the present invention commonly ranges from 0.1 to 10% by weight, preferably ranges from 0.5 to 5% by weight, and more preferably ranges from 0.5 to 5% by weight [sic].

In the preparation for external use on skin of the present invention, in addition to the components described above, surfactants, solubilizing components, fats and oils, sugars, or percutaneous absorption accelerator components can be further blended. In particular, by blending surfactants, solubilizing components, or fats and oils, stability of the water-soluble ascorbic acid in an aqueous medium, efficacy thereof, and sensation in use can be improved.

As examples of surfactants employed herein, mention may be made of various nonionic surfactants, examples of which include polyoxyethylene (hereinafter, referred to as POE) branched alkyl ethers such as POE octyldodecyl alcohol, POE 2-decyltetradecyl alcohol, and the like; POE alkyl ethers such as POE oleyl alcohol ether, POE cetyl alcohol ether, and the like; sorbitan esters such as sorbitan monooleate, sorbitan monoisostearate, sorbitan monolaurate, and the like; POE sorbitan esters such as POE sorbitan monooleate, POE sorbitan monoisostearate, POE sorbitan monolaurate, and the like; glycerol fatty acid esters such as glycerol monooleate, glycerol monostearate, glycerol monomyristate, and the like; POE glycerol fatty acid esters such as POE glycerol monooleate, POE glycerol monostearate, POE glycerol monomyristate, and the like; POE hardened castor oil fatty acid esters such as POE dihydrocholesterol ester, POE hardened castor oil, POE hardened castor oil isostearate, and the like; POE alkyl aryl ethers such as POE octyl phenyl ether, and the like; glycerol alkyl ethers such as monoisostearyl glyceryl ether, monomyristyl glyceryl ether, and the like; POE glycerol alkyl ethers such as POE monostearyl glyceryl ether, POE monomyristyl glyceryl ether, and the like; polyglycerol fatty acid esters such as diglyceryl monostearate, decaglyceryl decastearate, decaglyceryl decaisostearate, diglyceryl diisostearate, and the like; or natural surfactants such as lecithin, hydrogenated lecithin, saponin, surfactin sodium salt, cholesterol, bile acid, and the like; and the like. The surfactants described above can be employed alone or in combination of two or more kinds thereof.

In the case of employing the surfactants described above, the ratio thereof blended in the preparation for external use on skin of the present invention is not particularly limited as long as they do not provide adverse effects to the skin or mucosa and do not impair the effects of the present invention. They can be appropriately selected and employed in the ratio ranging from 0.01 to 30% by weight in the preparation for external use on skin of the present invention. In view of stability of active components in the preparation for external use on skin of the present invention, a sensation in use, and the like, the blending ratio thereof can preferably range from 0.1 to 20% by weight, and can more preferably range from 0.5 to 10% by weight. In addition, it is preferable that the surfactants be blended in an amount preferably ranging from 0.01 to 100 parts by weight with respect to 100 parts by weight of the glycol ether contained in the preparation for external use on skin of the present invention, and more preferably ranging from 0.01 to 50 parts by weight, and in particular, preferably ranging from 0.1 to 25 parts by weight. In addition, in view of stable solubilization of the water-soluble ascorbic acid in the preparation for external use on skin of the present invention, the blending ratio of the surfactants can range from 0.1 to 10 parts by weight with respect to 100 parts by weight of the water-soluble ascorbic acid, can preferably range from 0.1 to 5 parts by weight, and can more preferably range from 0.1 to 20 parts by weight [sic].

As fats and oils, they are not particularly limited as long as they are those employed as components of preparations for external use in the field of medicines, quasi drugs, or cosmetics. As examples thereof, mention may be made of synthetic oils such as middle-chain fatty acid triglyceride, and the like; vegetable oils such as soybean oil, rice oil, rapeseed oil, cotton seed oil, sesame oil, safflower oil, castor oil, olive oil, cacao butter, camellia oil, sunflower oil, palm oil, linseed oil, perilla oil, shea oil, saru [phonetic spelling] oil, coconut oil, Japan wax, jojoba oil, grape seed oil, avocado oil, and the like; animal oils such as mink oil, yolk oil, beef tallow, milk fat, lard, and the like; waxes such as beeswax, spermaceti wax, lanolin, carnauba wax, candelilla wax, and the like; hydrocarbons such as liquid paraffin, squalene, squalane, microcrystalline wax, ceresin wax, paraffin wax, vaseline, and the like; natural or synthetic fatty acids such as lauric acid, myristic acid, stearic acid, oleic acid, isostearic acid, behenic acid, and the like;-natural or synthetic higher alcohols such as cetanol, stearyl alcohol, hexyldecanol, octyldecanol, lauryl alcohol, and the like; esters or ethers such as isopropyl myristate, isopropyl palmitate, octyldodecyl myristate, octyldodecyl oleate, cholesterol oleate, and the like; silicone oils; and the like. The fats and oils described above can be employed alone or in combination of two or more kinds thereof.

In the case of employing the fats and oils described above, the ratio thereof blended in the preparation for external use on skin of the present invention is not particularly limited as long as they do not provide adverse effects to the skin or mucosa and do not impair the effects of the present invention. They can be appropriately selected and employed in the ratio ranging from 0.01 to 70% by weight in the preparation for external use on skin of the present invention. In view of stability of active components in the preparation for external use on skin of the present invention, sensation in use, and the like, the blending ratio thereof can preferably range from 0.1 to 60% by weight, and can more preferably range from 0.1. to 50% by weight. In addition, it is preferable that the fats and oils be blended in an amount preferably ranging from 0.0l to 1000 parts by weight with respect to 100 parts by weight of the glycol ether contained in the preparation for external use on skin of the present invention, and more preferably ranging from 0.01 to 800 parts by weight, and in particular, preferably ranging from 0.05 to 500 parts by weight. In addition, in view of improvement in stability of the water-soluble ascorbic acid, the blending ratio of the fats and oils can range from 0.1 to 1000 parts by weight with respect to 100 parts by weight of the water-soluble ascorbic acid, can preferably range from 0.1 to 500 parts by weight, and can more preferably range from 0.1 to 100 parts by weight.

As sugars, they are not particularly limited as long as they are those employed as components of preparations for external use in the field of medicines, quasi drugs, or cosmetics. As examples thereof, mention may be made of monosaccharides (such as glucose, galactose, mannose, ribose, arabinose, xylose, deoxyribose, fructose, ribulose, lyxose, and the like), disaccharides (such as sucrose, trehalose, lactose, maltose, cellobiose, and the like), oligosaccharides (such as lactulose, raffinose, pullulan, and the like), cellulose and derivatives thereof (such as methylcellulose, ethylcellulose, hydroxyethylcellulose, hydroxypropylcellulose, carboxymethylcellulose, carboxyethylcellulose, nitrocellulose, and the like), polymer sugars (such as chondroitin sulfate, hyaluronic acid, dermatan, heparan, heparin, keratan, and salts thereof (pharmaceutically or physiologically acceptable salts such as sodium chondroitin sulfate, sodium hyaluronate, dermatan sulfate, haparan sulfate, keratan sulfate, and the like), and the like), and sugar alcohols (such as mannitol, xylitol, erythritol, pentaerythritol, maltitol, sorbitol, polydextrose, and the like), and in addition, xylose, inositol, dextrin and derivatives thereof, honey, a muscovado extract, and the like. The sugars described above may be employed alone or in combination of two or more kinds thereof.

In the preparation for external use on skin of the present invention, various components which are generally employed as components of preparations for external use in the field of medicines, quasi drugs, or cosmetics, such as amino acids, reducers for irritation, thickening agents, preservatives, UV controlling agents, coloring agents, pH adjustors, perfumes, and the like, can be blended within a quantitative and qualitative range which does not impair the quality such as apparent stability, viscosity, and the like, and does not impair the effects of the present invention. The components described above can be freely employed alone or in combination of two or more kinds thereof.

The preparation for external use on skin of the present invention can be produced in the preferable form of a paste, a mousse, a gel, a liquid, a milky lotion, a cream, a sheet (base material carrier), an aerosol, a spray, or the like, by blending and mixing one or more kinds selected from the glycol, the glycol ether, the glycerol, and the diglycerol described above, the water-soluble ascorbic acid, and the low molecular weight betaine, and each of optional components described above, if necessary, and in addition, blending other solvents or base agents of preparations for external use generally employed, and the like. They can be produced in a conventional manner known in the art.

The preparation for external use on skin of the present invention may commonly have liquid properties at pH 1 to pH 8. In view of stability of the water-soluble ascorbic acid, low irritation with respect to the skin and mucosa, and a good sensation in use on the skin, it is preferably in the acidic range of preferably from pH 2 to pH 7, and more preferably from pH 2 to pH 6.

The preparations for external use on skin of the present invention can be formed into various compositions for external use in the field of cosmetics, medicines for external use, or quasi drugs for external use, such as makeup cosmetics such as foundations, lipsticks, mascaras, eye shadows, eyeliners, eyebrow colors, nail varnishes, and the like; base cosmetics such as milky lotions, creams, lotions, oils, packs, and the like; cleansing compositions such as face cleansing compositions, cleansers, body washes, and the like; underarm deodorants, athlete's foot remedies, anti-itching preparations, wound healing preparations, dry bathing preparations, cleaning preparations, anti-inflammatory analgesic preparations, acne remedies, hemorrhoidal preparations, sterilizing preparations, whitening preparations, UV controlling preparations, and the like. In view of effects on the skin, the preparations of the present invention are preferably employed as productions for applying on the outer skin, such as preparations for external use on skin (pharmaceutical preparations for outer skin) and the like.

In addition, the present invention also includes a method for stabilizing a water-soluble ascorbic acid. In the method of the present invention, stabilization of the water-soluble ascorbic acid can be achieved by employing a water-soluble ascorbic acid and one or more kinds selected from a glycol, a glycol ether, a glycerol, and a diglycerol, together with a low molecular weight betaine.

In the method of the present invention, the kinds of the water-ascorbic acids, glycols, glycol ethers, glycerols, diglycerols, and low molecular weight betaines, and the amounts thereof are the same as those employed in the preparations for external use on skin described above. In addition, the preparations produced in accordance with the method of the present invention can be employed in accordance with known or conventional dosage and administration one or more times by dividing the dose over a day.

### EXAMPLES

In the following, the present invention is described in detail based on Examples and Test Examples. It should be understood that the present invention is not limited to the Examples and the like. In each of the composition examples described below, “%” means % by weight (W/W), unless otherwise indicated.

### Test Example 1

### Tests on Solubilization, Sensation in Use, and Residual Ratio of Ascorbic Acids

In accordance with the compositions (% by weight) described in Table 1, preparations were prepared by adding ascorbic acid to a mixed solution of trimethylglycine, diethylene glycol monoethyl ether, 1,3-butylene glycol, polyoxyethylene (60) hardened castor oil, and water, and dissolving the mixture by heating and mixing the mixture.

Solubilization

Each of the preparations was allowed to stand for one month at 4° C. and at room temperature. Subsequently, presence or absence of crystal deposition was evaluated by visual observation of each of the test liquids.

Sensation in Use and Effect Impression

The sensation in use when each of the preparations immediately after preparation was applied to the skin and the effect impression after each of the preparations was employed for one month were evaluated with respect to the evaluation categories described below by 30 subjects.

Sensation in use: spreadability, fitness to the skin, non-smoothness of the preparation during application Effect impression: whitening effects such as fading spots, reducing pigmentation irregularities, and the like; skin elasticity effects such that wrinkles became inconspicuous, skin pores became invisible, and the like; and humectant effects such as smoothening the surface of the skin, and the like.

The sensation in use and the effect impression based on the evaluation categories described above were totally evaluated respectively at 5 levels of satisfaction, slight satisfaction, normal, slight dissatisfaction, and dissatisfaction. The case in which 20 or more subjects evaluated as satisfaction or slight satisfaction was indicated as ◯; the case in which 10 to 19 subjects evaluated as satisfaction or slight satisfaction was indicated as Δ; and the case in which not more than 9 subjects evaluated as satisfaction or slight satisfaction was indicated as ×.

Residual Ratio

The preparations of Example 1 and Comparative Example 4 were allowed to stand for 2 weeks at 50° C. Subsequently, a residual ratio (%) of ascorbic acid was measured.

The results are shown in Table 1.

TABLE 1

| | | | | Comparative | Comparative | Comparative | Comparative |
|---|---|---|---|---|---|---|---|
| | | Example 1 | Example 2 | Example 1 | Example 2 | Example 3 | Example 4 |
| L-ascorbic acid | | 5 | 20 | 5 | 20 | 20 | 20 |
| Trimethylglycine | | 5 | 3 | — | — | — | — |
| Polyoxyethylene (60) | | — | — | — | — | 3 | — |
| hardened castor oil | | | | | | | |
| Diethylene glycol monoethyl | | — | 57 | — | 60 | 57 | — |
| ether | | | | | | | |
| 1,3-butylene glycol | | 90 | — | 95 | — | — | — |
| Purified water | | — | 20 | — | 20 | 20 | 80 |
| Solubilization | Stored for | ◯ | ◯ | X | X | — | X |
| | one month | | | | | | |
| | at 4° C. | | | | | | |
| | Stored for | ◯ | ◯ | X | X | — | ◯ |
| | one month | | | | | | |
| | at room | | | | | | |
| | temperature | | | | | | |

| | | | | | | |
|---|---|---|---|---|---|---|
| Sensation in use (total | Δ | ◯ | X | X | — | ◯ |
| evaluation) | | | | | | |
| Effect impression (total | ◯ | ◯ | X | X | — | X |
| evaluation) | | | | | | |
| Residual ratio (%) | 99.1 | 97.5 | — | — | — | 60.2 |

In Comparative Examples 1 and 2, due to the poor sensation in use and deposition over time, storage tests for one month could not be carried out. In addition, in Comparative Example 3, in spite of blending a surfactant, ascorbic acid could not be dissolved during preparation, and therefore, a preparation could not be formed. In addition, in ° Comparative Example 4, a poor effect impression was provided, crystals were deposited at 4° C., and the residual ratio was extremely reduced. On the other hand, in Examples 1 and 2, the satisfactory solubilization, the satisfactory sensation in use, the satisfactory effect impression, and the satisfactory residual ratio could be obtained.

As described above, it was confirmed that the preparations of the present invention comprising a low molecular weight betaine could stably dissolve an acidic organic compound such as a water-soluble ascorbic acid, a superior sensation in use and a superior effect impression were provided, and a high residual ratio of a water-soluble ascorbic acid could be obtained.

In the following, Examples of preparations are described. The blending amounts in the Examples described below are based on % by weight unless otherwise indicated.

### Example 3

| | |
|---|---|
| Ascorbic acid | 20 |
| Trimethylglycine | 3 |
| Diethylene glycol monoethyl ether | 52.4 |
| Diglycerol | 4 |
| Purified water | 20 |
| Perfume | 0.5 |
| Grapefruit extract | 0.1 |
| Total | 100% |

### Example 4

| | |
|---|---|
| Ascorbic acid | 10 |
| Trimethylglycine | 1 |
| Diethylene glycol monoethyl ether | 84.7 |
| Diglycerol | 4 |
| Perfume | 0.3 |
| Total | 100% |

### Example 5

| | |
|---|---|
| Ascorbic acid | 8 |
| Trimethylglycine | 5 |
| 1,3-butylene glycol | 86.4 |
| Lemon extract | 0.1 |
| Perfume | 0.5 |
| Total | 100% |

### Example 6

| | |
|---|---|
| Ascorbic acid | 10 |
| Trimethylglycine | 3 |
| Diethylene glycol monoethyl ether | 65.4 |
| Propylene glycol | 20 |
| Diglycerol | 1 |
| Acerola extract | 0.1 |
| Perfume | 0.5 |
| Total | 100% |

### Example 7

| | |
|---|---|
| Ascorbic acid | 15 |
| Trimethylglycine | 3 |
| Diethylene glycol monoethyl ether | 50.9 |
| Propylene glycol | 20 |
| Purified water | 10 |
| Diglycerol | 1 |
| Acerola extract | 0.1 |
| Total | 100% |

### Example 8

| | |
|---|---|
| Ascorbic acid | 30 |
| Trimethylglycine | 9 |
| Diethylene glycol monoethyl ether | 30.9 |
| Purified water | 30 |
| Orange extract | 0.1 |
| Total | 100% |

### Example 9

| | |
|---|---|
| Ascorbyl 2-glucoside | 20 |
| Trimethylglycine | 3 |
| Diethylene glycol monoethyl ether | 52.4 |
| Diglycerol | 4 |
| Purified water | 20 |
| Perfume | 0.5 |
| Grapefruit extract | 0.1 |
| Total | 100% |

### Example 10

| | |
|---|---|
| Ascorbyl 2-sulfuric ester | 10 |
| Trimethylglycine | 1 |
| Diethylene glycol monoethyl ether | 84.7 |
| Diglycerol | 4 |
| Perfume | 0.3 |
| Total | 100% |

### Example 11

| | |
|---|---|
| Sodium ascorbate | 8 |
| Trimethylglycine | 5 |
| 1,3-butylene glycol | 86.4 |
| Lemon extract | 0.1 |
| Perfume | 0.5 |
| Total | 100% |

### Example 12

| | |
|---|---|
| Ascorbyl monophosphoric ester | 10 |
| Trimethylglycine | 3 |
| Diethylene glycol monoethyl ether | 65.4 |
| Propylene glycol | 20 |
| Diglycerol | 1 |
| Acerola extract | 0.1 |
| Perfume | 0.5 |
| Total | 100% |

### Example 13

| | |
|---|---|
| Sodium ascorbyl monophosphate | 15 |
| Trimethylglycine | 3 |
| Diethylene glycol monoethyl ether | 50.9 |
| Propylene glycol | 20 |
| Purified water | 10 |
| Diglycerol | 1 |
| Acerola extract | 0.1 |
| Total | 100% |

### Example 14

| | |
|---|---|
| Sodium ascorbyl 2-sulfate | 30 |
| Trimethylglycine | 9 |
| Diethylene glycol monoethyl ether | 30.9 |
| Purified water | 30 |
| Orange extract | 0.1 |
| Total | 100% |

What is claimed is:

1. A preparation for external use on skin, comprising:
(A) ascorbic acid, in an amount of not less than 3% by weight;
(B) one or more compounds selected from a glycol, a glycol ether, a glycerol, and a diglycerol; and
(C) a low molecular weight betaine.

2. The preparation for external use on skin according to claim 1, wherein the low molecular weight betaine is trimethylglycine.

3. A preparation for external use on skin, comprising:
(A) a water-soluble ascorbic acid;
(B) one or more compounds selected from a glycol, a glycol ether, a glycerol, and a diglycerol; and
(C) a solubilizer consisting of a low molecular weight betaine.

4. A method for stabilizing a water-soluble ascorbic acid, comprising a step of employing a low molecular weight betaine together with a preparation for external use on skin comprising:
(A) a water-soluble ascorbic acid; and
(B) one or more compounds selected from a glycol, a glycol ether, a glycerol, and a diglycerol.
